(19) United States
(12) Patent Application Publication
(10) Pub. No.: US 2025/0163383 A1
(43) Pub. Date: May 22, 2025

(54) CELL CULTURE MEMBRANE AND ORGAN IMITATION STRUCTURE

(71) Applicant: TOYODA GOSEI CO., LTD., Kiyosu-shi (JP)

(72) Inventors: Sayaka MIYATA, Aichi (JP); Yasuhiko SHINODA, Aichi (JP); Masateru YAMAZAKI, Aichi (JP); Seitaro TAKI, Aichi (JP); Seiichi ISHIDA, Aichi (JP); Taku MATSUSHITA, Aichi (JP); Satoha AIHOSHI, Aichi (JP)

(21) Appl. No.: 18/947,127

(22) Filed: Nov. 14, 2024

(30) Foreign Application Priority Data
Nov. 16, 2023 (JP) 2023-195291

(51) Int. Cl. C12N 5/071; C12M 1/12

(52) U.S. Cl. CPC C12N 5/0671; C12M 25/04; C12N 2533/30

(57) ABSTRACT

A cell culture membrane, which is capable of culturing a hepatic stellate cell, includes: a membrane body having a first face and a second face located on a side opposite to the first face; and plural holes provided in the membrane body, each of the plural holes has a mortar shape, and is open to at least the first face, and a first average hole diameter of the plural holes at the first face is 4 μm or more and 13 μm or less.

### CROSS-REFERENCE TO RELATED APPLICATION

This application is based on and claims priority under 35 USC 119 from Japanese Patent Application No. 2023-195291 filed on Nov. 16, 2023.

### TECHNICAL FIELD

The present disclosure relates to a cell culture membrane and an organ imitation structure.

### BACKGROUND ART

In the conventional art, attempts have been made to produce a human liver model for, for example, drug discovery research. It is known that human hepatic stellate cells change from a deactivated state to an activated state. In an experiment, the human hepatic stellate cells are preferably in the deactivated state. For example, WO2020/171220 describes a method for producing a human liver-like three-dimensional structure including heterospheroids obtained by aggregating human liver cells and human hepatic stellate cells. WO2020/171220 suggests use of a stellate cell activation inhibitor for preventing a change of the human hepatic stellate cells to an activated state during production of the human liver-like three-dimensional structure.

### SUMMARY OF INVENTION

As a method other than the production of spheroids, attempts have been made to produce a human liver model by co-culture using the cell culture membrane. When hepatic stellate cells are two-dimensionally cultured using the cell culture membrane, hepatic stellate cells tend to change from a deactivated state to an activated state.

The present disclosure can be implemented by the following aspects.

(1) According to a first aspect of the present disclosure, a cell culture membrane is provided. The cell culture membrane is a cell culture membrane capable of culturing a hepatic stellate cell and includes: a membrane body having a first face and a second face located on a side opposite to the first face; and a plurality of holes formed in the membrane body, in which each of the plurality of holes has a mortar shape and is open to at least the first face, and a first average hole diameter of the plurality of holes at the first face is 4 μm or more and 13 μm or less. According to this aspect, since each of the plurality of holes has the mortar shape, the hepatic stellate cell is cultured in a state in which the hepatic stellate cell has entered the plurality of holes. Further, by using the cell culture membrane having the first average hole diameter of 4 μm or more and 13 μm or less, it is possible to prevent a change from a deactivated state to an activated state and culture the hepatic stellate cell.

(2) In the cell culture membrane of the above aspect, the first average hole diameter may be 6 μm or more and 10 μm or less. According to this aspect, it is possible to further prevent the change of the hepatic stellate cell from the deactivated state to the activated state.

(3) In the cell culture membrane of the above aspect, each of the plurality of holes may be a through hole penetrating from the first face to the second face. According to this aspect, when the hepatic stellate cell is cultured on the first face and the cell different in type from the hepatic stellate cell is cultured on the second face, since the hepatic stellate cell cultured on the first face and the cell cultured on the second face can come into contact with each other through each of the through holes, an organ imitation structure closer to a state in the living body can be created.

(4) The organ imitation structure of the above aspect may include: the cell culture membrane according to the above aspect; and the hepatic stellate cell cultured on the first face. According to this aspect, the cultured hepatic stellate cell provided in the organ imitation structure is prevented from changing from the deactivated state to the activated state. Thus, a toxicity test of drugs or the like can be suitably performed using the organ imitation structure.

(5) An organ imitation structure may include: the cell culture membrane according to the above aspect; the hepatic stellate cell cultured on the first face; and a hepatocyte cultured on the second face. According to this aspect, the hepatic stellate cell and the hepatocyte are in contact with each other via the holes. Thus, by using the organ imitation structure, it is possible to evaluate interaction caused by an intercellular contact between the hepatocyte and the hepatic stellate cell in the deactivated state after the addition of the substances.

(6) According to a second aspect of the present disclosure, there is provided a cell culture membrane capable of culturing a hepatic stellate cell. The cell culture membrane is formed of polyurethane, and when the hepatic stellate cell is cultured on the cell culture membrane, a deactivated state of the hepatic stellate cell is maintained for one week or more. According to this aspect, the hepatic stellate cell cultured in the cell culture membrane is prevented from changing from the deactivated state to the activated state. Accordingly, an experiment for drug discovery can be suitably performed.

The present disclosure can be implemented in various aspects, and can be implemented in the aspect of, for example, a manufacturing method of a cell culture membrane in addition to the above cell culture membrane.

### BRIEF DESCRIPTION OF DRAWINGS

FIG. 1 is a schematic view of a cross section of a cell culture membrane.

FIG. 2 is a schematic view of a first organ imitation structure.

FIG. 3 is a schematic view of a second organ imitation structure.

FIG. 4 is a diagram illustrating a method for producing the organ imitation structure.

FIG. 5 is a diagram showing another embodiment of the cell culture membrane.

FIG. 6 is a table summarizing cell culture membranes and evaluation results of states of cultured cells.

### DESCRIPTION OF EMBODIMENTS

### A. Embodiment

It is known that hepatic stellate cells change from a deactivated state to an activated state. The hepatic stellate cells are usually present in the deactivated state in a living body, but are activated by chronic inflammation or injuries. The activated hepatic stellate cells are transformed into myofibroblast-like cells with different functions and forms, and synthesize excess collagen. It is estimated that the activated hepatic stellate cells cause hepatic fibrosis and hepatic cirrhosis. Thus, the hepatic stellate cells in the deactivated state are required for the study of the hepatic cirrhosis, for example. However, in general, in vitro culture, the hepatic stellate cells tend to change from the deactivated state to the activated state. The present inventors have found a cell culture membrane capable of preventing a change of the hepatic stellate cell to the activated state. The deactivated state is also referred to as a stationary state or an inactive state.

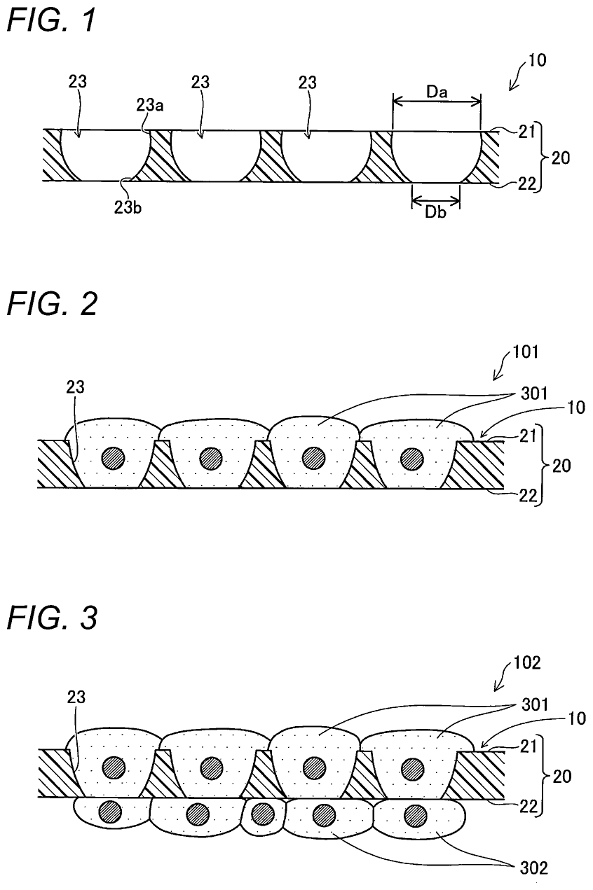

FIG. 1 is a schematic view of a cross section of a cell culture membrane 10. The cell culture membrane 10 is capable of culturing the hepatic stellate cells. The cell culture membrane 10 includes a membrane body 20 and a plurality of holes 23 formed in the membrane body 20. The cell culture membrane 10 is formed of a thermosetting resin. Polyurethane, polyurea, a silicone resin, a phenol resin, an epoxy resin, an unsaturated polyester, polyimide, and the like can be used as the thermosetting resin forming the cell culture membrane 10. In the present embodiment, the cell culture membrane 10 is formed of polyurethane. When culturing hepatic stellate cells 301, the cell culture membrane 10 is maintained in a deactivated state of the hepatic stellate cells 301 for one week or more. The membrane body 20 has a first face 21 and a second face 22 located on a side opposite to the first face 21. In the present embodiment, the plurality of holes 23 are through holes penetrating from the first face 21 to the second face 22. Each hole 23 has a first opening portion 23a opening to the first face 21 and a second opening portion 23b opening to the second face 22. Since the cell culture membrane 10 has the second opening portions 23b, when the hepatic stellate cells are cultured on the first face 21 and cells different in type from the hepatic stellate cells are cultured on the second face 22, the hepatic stellate cells and the cells different from the hepatic stellate cells can be brought into contact with each other through the second opening portions 23b.

The hole 23 has a mortar shape. In the case of the through holes, the term “mortar shape” used herein encompasses a shape in which a second hole diameter Db, which is the diameter of the second opening portion 23b, is smaller than a first hole diameter Da, which is the diameter of the first opening portion 23a. Further, both a case where the shape of an inner peripheral portion connecting the first opening portion 23a to the second opening portion 23b is a curved face and a case where the shape of the inner peripheral portion is a plane are encompassed by the term “mortar shape” used herein. A case where the shape of an inner peripheral portion connecting the first opening portion 23a to the second opening portion 23b includes both of a curved face and a vertical plane so that a cross-sectional shape of the hole 23 is like U-shape is also encompassed by the term “mortar shape” used herein. A second average hole diameter, which is an average value of the second hole diameters Db at the second face 22, is smaller than a first average hole diameter, which is an average value of the first hole diameters Da of the plurality of holes 23. Accordingly, the hepatic stellate cells 301 can be cultured in a state in which they have entered the inside of the holes 23. The first average hole diameter is preferably 4 μm or more and 13 μm or less, and more preferably 6 μm or more and 10 μm or less. Accordingly, when the hepatic stellate cells 301 shown in FIG. 2 are cultured on the first face 21 of the cell culture membrane 10, it is possible to prevent the change of the hepatic stellate cells 301 from the deactivated state to the activated state. When the cell culture membrane 10 is used, since the deactivated state of the hepatic stellate cells 301 can be maintained for one week or more, for example, a test substance can be exposed to the cultured hepatic stellate cells 301 for a long period of time or repeatedly, and prediction accuracy of long-term toxicity is improved.

The first average hole diameter is a value obtained by irradiating the upward-facing first face 21 with light and performing observation with a microscope in a state in which the first face 21 is on the top and the second face 22 is on the bottom. Since the light is not reflected from the hole 23, the hole 23 is visually recognized in black. Specifically, circle equivalent diameters of all the holes 23 observed in a specific visual field are measured, and the first average hole diameter is an average value of the measured values. The circle equivalent diameter refers to the diameter of a true circle corresponding to the area of the hole 23. The second average hole diameter is similarly obtained. That is, the value is obtained by irradiating the upward-facing second face 22 with light and performing observation with a microscope in a state in which the second face 22 is on the top and the first face 21 is on the bottom.

The cell culture membrane 10 is manufactured by supplying water vapor to an uncured polyurethane raw material formed in a thin plate shape on a membrane forming substrate, and foaming and curing the uncured polyurethane raw material. According to this manufacturing method, since the hole 23 is formed by foaming, the cell culture membrane 10 in which the first hole diameter Da and the second hole diameter Db are different from each other can be easily manufactured. For example, manufacturing may be performed as in JP6343492B.

FIG. 2 is a schematic diagram of an organ imitation structure 101. As shown in FIG. 2, the organ imitation structure 101 includes the cell culture membrane 10 and the hepatic stellate cells 301 cultured on the first face 21. The cultured hepatic stellate cells 301 are prevented from changing from the deactivated state to the activated state. Thus, a toxicity test of drugs or the like can be suitably performed using the organ imitation structure 101. FIG. 3 is a schematic diagram of an organ imitation structure 102. As shown in FIG. 3, the organ imitation structure 102 includes the cell culture membrane 10, the hepatic stellate cells 301 cultured on the first face 21, and hepatocytes 302 cultured on the second face 22. The cultured hepatic stellate cells 301 are prevented from changing from the deactivated state to the activated state. The hepatic stellate cells 301 and the hepatocytes 302 are in contact with each other via the holes 23. Thus, by using the organ imitation structure 102, it is possible to evaluate interaction caused by an intercellular contact between the hepatocytes 302 and the hepatic stellate cells 301 in the deactivated state after the addition of the substances. By using the organ imitation structure 102, liver fibrosis and activation after addition of a liver damage factor can be evaluated.

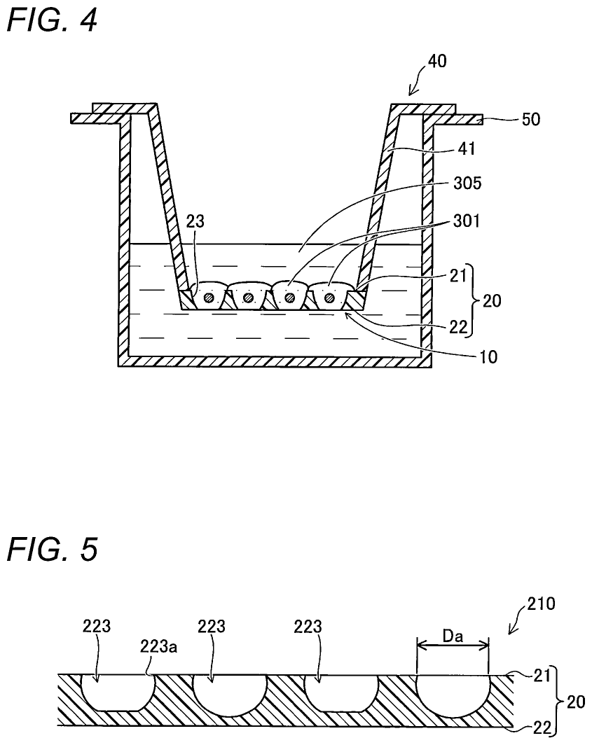

FIG. 4 is a diagram illustrating a method for producing the organ imitation structure 101 and the organ imitation structure 102. When the organ imitation structure 101 and the organ imitation structure 102 are produced, an insert 40 shown in FIG. 4 is used. The insert 40 includes an insert body portion 41 made of a resin and the cell culture membrane 10. The insert body portion 41 has a generally cylindrical shape. The cell culture membrane 10 is attached to one end portion of the insert body portion 41 in an axial direction. A flange portion extending in a direction orthogonal to the axial direction is formed at the other end portion of the insert body portion 41 in the axial direction.

When the hepatic stellate cells 301 are cultured using the insert 40, the insert 40 is inserted into a well 50. Since flange portions of the insert 40 come into contact with the well 50, the cell culture membrane 10 is maintained in a state away from the well 50. Then, the hepatic stellate cells 301 are seeded and cultured on the first face 21 in a state in which a culture solution 305 is put in the wells 50 to the extent that the cell culture membrane 10 is immersed.

When the organ imitation structure 102 is produced, the hepatocytes 302 are cultured on the second face 22 after the hepatic stellate cells 301 are cultured on the first face 21 by the above method. Specifically, when the hepatocytes 302 are cultured on the second face 22, the insert 40 is reversed such that the second face 22 is positioned above the first face 21. Next, in a state in which the cell culture membrane 10 is immersed in the culture solution 305, the hepatocytes 302 are seeded and cultured on the second face 22. The method for producing the organ imitation structure 102 is not limited thereto, and the hepatic stellate cells 301 may be cultured on the first face 21 after the hepatocytes 302 are cultured on the second face 22. Immune cells may be further cultured on the hepatic stellate cells 301. In the production of the organ imitation structure 102 and the use thereof, since various operations are performed on each of the plurality of cells, it is desirable for the cells to be maintained in the deactivated state for a longer period of time than when each of the cells is cultured alone. The use of the cell culture membrane 10 is preferable since the deactivated state of the hepatic stellate cells 301 can be maintained for one week or more. In particular, after the culturing of the hepatic stellate cells 301, the deactivation can be maintained even while a different type of cell is seeded and cultured on the second face 22, which is preferable.

### B. Another Embodiment of Cell Culture Membrane

FIG. 5 is a schematic view showing a cell culture membrane 210 which is another embodiment of the cell culture membrane 10. The same configurations as those of the above embodiment are denoted by the same reference numerals, and detailed description thereof will be appropriately omitted. As shown in FIG. 5, the cell culture membrane 210 is different from the above cell culture membrane 10 in that each of a plurality of holes 223 is a non-through hole that does not open to the second face 22.

The hole 223 has the mortar shape as in the above embodiment. In the case of the non-through hole, the following shapes (A) and (B) are encompassed by the term “mortar shape” used herein. The shape (A) refers to a shape in which an inner peripheral face defining the hole 223 is substantially hemispherical with a vertex at a point where a distance between this inner peripheral face and the second face 22 is the shortest (In the shape (A), the inner peripheral face may be a curved face or may be a combination of a curved face and a vertical plane so that a cross-sectional shape of the hole 223 is like U-shape.). The shape (B) refers to a shape in which at a position where the distance between the inner peripheral face defining the hole 223 and the second face 22 is the shortest, an area of a cross section of the inner peripheral face parallel to the first face 21 is smaller than an area of a first opening portion 223a of the hole 223 at the first face 21. Even when the hole 223 is the non-through hole, activation of the hepatic stellate cells 301 cultured on the first face 21 is prevented.

### C. Experimental Results

### C-1. Cell Culture Membranes

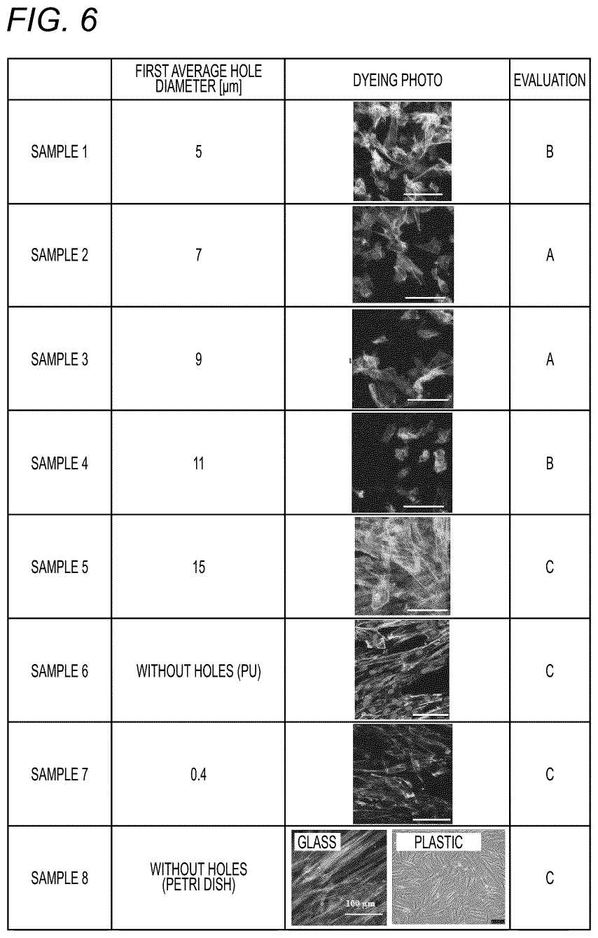

FIG. 6 is a table summarizing the cell culture membranes and evaluation results of states of cultured cells. Samples 1 to 5 are cell culture membranes produced by supplying water vapor to an uncured polyurethane raw material formed in a thin plate shape on a membrane forming substrate and foaming and curing the uncured polyurethane raw material. By adjusting a reaction temperature, a reaction time, and a composition of the uncured polyurethane raw material, which are conditions for producing polyurethane, Samples 1 to 5 were produced, which are cell culture membranes with various diameters of the through holes formed in the cell culture membranes. The through hole has the mortar shape, and is open to the first face and the second face of the membrane body of the cell culture membrane. The hole diameter at the first face is larger than the hole diameter at the second face. The first average hole diameter shown in FIG. 6 was obtained in a manner similar to that in the above embodiment. A membrane thickness of Samples 1 to 5 is about 5 μm.

Sample 6 is a cell culture membrane of polyurethane without holes. Sample 6 was produced by curing the uncured polyurethane raw material without supplying the water vapor to the uncured polyurethane raw material formed in the thin plate shape on the membrane forming substrate. Sample 7 is a resin membrane manufactured by Transwell Co., Ltd. Specifically, the product name of Sample 7 is Transwell Clear 24 Well 0.4 μm, and the cell culture surface is treated and sterilized. The average hole diameter of holes formed in Sample 7 is 0.4 μm. A membrane thickness of Sample 7 is about 10 μm. Sample 8 is a Petri dish. Sample 8 includes a plastic Petri dish and a glass bottom Petri dish.

### C-2. Cell Culture

Cells were cultured using Samples 1 to 8. Cell culture conditions are as follows.
Cell name: LI90
Passage number: 25
Sowing density: 1.5×10⁵ cells/cm² (5.0×10⁴ cells/well)

The LI90 cell is a cell derived from a human hepatic mesenchymal cell tumor and having a feature similar to that of the hepatic stellate cell. Hereinafter, each of scaffolds of Samples 1 to 8 and a cell cultured on the scaffold will be referred to as a sample.

### C-3. Cell Observation Method

The following first to fourth steps were performed in this order. As the first step, the medium was changed after 3 days from the sowing, and the cells were fixed after 7 days from the cell sowing. A specific fixation method was as follows: the medium was removed, the cells were washed twice with 0.5 mL of PBS (−), and then 0.5 mL of a 10% neutral buffered formalin solution was added, and the cells were fixed at room temperature for 15 minutes. After completion of the fixation, the sample was washed three times with 0.5 mL of PBS (−). In the case of storing the sample, the supernatant was removed, a buffer solution was added, and the sample was stored at a temperature of 4° C. in the dark.

As the second step, 0.5 mL of 0.1% Triton X-100 was added to perform membrane permeation treatment for 15 minutes. After the membrane permeation treatment, the sample was washed twice with 0.5 mL of PBS (−).

As the third step, a phalloidin staining solution (1/40) was prepared, and 400 μL of the staining solution was added to the sample. After the addition, a lid was covered to prevent evaporation, and the sample was incubated at a room temperature for 60 minutes. After phalloidin staining, the sample was washed twice with 0.5 mL of PBS (−), and 0.5 mL of TO-PRO-3 (1/1000) was added and reacted for 10 minutes to perform nuclear staining.

In the fourth step, the sample was washed twice with 0.5 mL of PBS (−), and two drops of an anti-fading agent were added onto the sample. Thereafter, the cells were observed with a confocal laser microscope.

### C-4. Cell Observation Result

In the cells after being stained, actin (F-actin) is stained green, and the nucleus is stained red. In a monochrome observation image shown in FIG. 6, actin is shown in gray. The actin of the LI 90 cell in the activated state is fibrosis. By observing the shape of the actin, an activation prevention effect of the LI90 cell was evaluated.

As shown in Samples 6 and 8 in FIG. 6, actin fibers extend in the cell culture membrane without holes and the Petri dish. As shown in Sample 7, even in the cell culture membrane with the holes formed therein, in the cell culture membrane in which the hole diameter is less than 1 μm, the actin fibers extend. As shown in Sample 5, even in the cell culture membrane with holes formed therein, when the hole diameter is 15 μm or more, the actin fibers extend. On the other hand, as shown in Samples 1 to 4, in the cell culture membranes having a first average hole diameter of 5 μm or more and 11 μm or less, a proportion of cells in which the actin was not fibrotic was high.

In a specific visual field of the microscope, the activation prevention effect was evaluated and ranked into three levels based on a ratio of the cells with extended actin fibers to all the cells observed. “Evaluation” in FIG. 6 shows evaluation results. Specifically, the three levels are “A”, “B”, and “C”. The cells were evaluated and ranked into “A”, “B,” and “C” in order of decreasing percentage of cells with extended actin fibers. That is, “A” indicates that the activation prevention effect of the cells is the highest. As shown in FIG. 6, in order to prevent the activation of the cells, the first average hole diameter is preferably 4 μm or more and 13 μm or less, and more preferably 6 μm or more and 10 μm or less. The size of the nucleus of the cultured cell is about 5 μm. It is considered that when the hole diameter is 4 μm or more and 13 μm or less, the cells are cultured in a state in which they have entered the holes, so that the cells are less likely to be stressed, and the activation is prevented. Further, it is considered that when the hole diameter is large, a plurality of cells enter the holes, the cells are less likely to be stressed, and the cells are easily activated. Although an observation image in which the hole diameter is less than 5 μm is not shown, in Sample 1, cells in which the action fibers do not extend are observed. Further, the size of the nucleus of the cell is about 5 μm. It is considered that when the hole diameter is at least 4 μm or more, since the cells are cultured in a state in which at least a portion of the cells has entered the holes, the cells are less likely to be stressed, and the activation can be prevented. In the above experiment, LI90 cells, which are human-derived cells, are used. However, when the cell culture membrane 10 is used, the cells in which the activation prevention effect is exerted are not limited to those derived from humans. For example, cells derived from rodents such as rats and mice can also exert the activation prevention effect since the cells are less likely to be stressed, similar to the cells derived from humans.

According to the embodiment described above, the cell culture membrane 10 includes the membrane body 20 and the plurality of holes 23 formed in the membrane body 20. Each of the plurality of holes 23 has the mortar shape and opens at least to the first face 21. The first average hole diameter at the first face 21 of the plurality of holes 23 is 4 μm or more and 13 μm or less. Since each of the plurality of holes 23 has the mortar shape, when the hepatic stellate cells are cultured on the first face 21, the hepatic stellate cells are cultured in a state in which they have entered the plurality of holes 23. Since the first average hole diameter 4 μm or more and 13 μm or less, the hepatic stellate cells can be cultured while preventing the change from the deactivated state to the activated state. Further, when the first average hole diameter is 6 μm or more and 10 μm or less, it is possible to further prevent the change of the hepatic stellate cells from the deactivated state to the activated state.

Further, each of the plurality of holes 23 is a through hole penetrating from the first face 21 to the second face 22. When the hepatic stellate cells 301 are cultured on the first face 21 and the cells different in type from the hepatic stellate cells 301 are cultured on the second face 22, since the hepatic stellate cells 301 cultured on the first face 21 and the cells cultured on the second face 22 can come into contact with each other through the holes, the organ imitation structure closer to a state in the living body can be created.

The organ imitation structure 101 includes the cell culture membrane 10 and the hepatic stellate cells 301 cultured on the first face 21. The cultured hepatic stellate cells 301 provided in the organ imitation structure 101 are prevented from changing from the deactivated state to the activated state. Thus, the toxicity test of drugs or the like can be suitably performed using the organ imitation structure 101. The organ imitation structure 102 also includes the hepatic stellate cells 301 cultured on the first face 21 and the hepatocytes 302 cultured on the second face 22. Thus, by using the organ imitation structure 102, it is possible to evaluate interaction caused by an intercellular contact between the hepatocytes and the hepatic stellate cells in the deactivated state after the addition of the substances.

In the embodiment described above, another element for preventing the change of the hepatic stellate cells 301 to the activated state, for example, a stellate cell activation inhibitor, is not used in the culture. When the stellate cell activation inhibitor is used, the interaction such as inhibition of a cell function by the stellate cell activation inhibitor and activation by the stellate cell activation inhibitor may affect the state of the cell. In this regard, by using the cell culture membrane 10, it is possible to prevent the change of the hepatic stellate cells 301 to the activated state without using the stellate cell activation inhibitor. Thus, it is possible to evaluate the change of the cells to the activation state caused by evaluation targets such as drugs and environments, in a state where the interaction caused by the above stellate cell activation inhibitor is eliminated.

The present disclosure is not limited to the above-described embodiments, and may be implemented by various configurations without departing from the gist of the present disclosure. For example, technical features in the embodiments corresponding to technical features in aspects described in the summary of the invention can be replaced or combined as appropriate to solve a part or all of the above-described problems or to achieve a part or all of the above-described effects. The technical features may be appropriately deleted unless being described as necessary in the present specification.

### REFERENCE SIGNS LIST

10, 210 cell culture membrane
20 membrane body
21 first face
22 second face
23, 223 hole
23a, 223a first opening portion
23b second opening portion
40 insert
41 insert body portion
50 well
101, 102 organ imitation structure
301 hepatic stellate cell
302 hepatocyte
305 culture solution
Da first hole diameter
Db second hole diameter

What is claimed is:

1. A cell culture membrane capable of culturing a hepatic stellate cell, the membrane comprising:
a membrane body having a first face and a second face located on a side opposite to the first face; and
a plurality of holes provided in the membrane body, wherein
each of the plurality of holes has a mortar shape, and is open to at least the first face, and
a first average hole diameter of the plurality of holes at the first face is 4 μm or more and 13 μm or less.

2. The cell culture membrane according to claim 1, wherein
the first average hole diameter is 6 μm or more and 10 μm or less.

3. The cell culture membrane according to claim 1, wherein
each of the plurality of holes is a through hole penetrating from the first face to the second face.

4. An organ imitation structure comprising:
the cell culture membrane according to claim 1; and
the hepatic stellate cell cultured on the first face.

5. An organ imitation structure comprising:
the cell culture membrane according to claim 1;
the hepatic stellate cell cultured on the first face; and
a hepatocyte cultured on the second face.

6. A cell culture membrane capable of culturing a hepatic stellate cell, wherein
the cell culture membrane comprises polyurethane, and
when the hepatic stellate cell is cultured on the cell culture membrane, a deactivated state of the hepatic stellate cell is maintained for one week or more.
